(19) United States
(12) Patent Application Publication
(10) Pub. No.: US 2017/0173319 A1
(43) Pub. Date: Jun. 22, 2017

(54) ROTARY TATTOO MACHINE AND RETURN SPRING

(71) Applicant: Eikon Device Inc., Kingston (CA)

(72) Inventors: Mike McGuire, Ottawa (CA); Charles Anthony Reid, Kingston (CA)

(21) Appl. No.: 15/237,927

(22) Filed: Aug. 16, 2016

(30) Foreign Application Priority Data
Dec. 18, 2015 (CA) 2915552

(51) Int. Cl. A61M 37/00; A61B 17/34

(52) U.S. Cl. CPC A61M 37/0076; A61B 17/3476; A61B 17/34

(57) ABSTRACT

A tattoo machine having a body adapted to receive a return spring. The body of the tattoo machine defines a channel which is configured to selectively receive the return spring. The channel is defined through a bottom surface of the tattoo machine body and may be closed on the bottom surface by a cap or retaining means. The return spring may be inserted, removed or replaced without disassembling the body, motor and slide components of the machine. The tattoo machine may be configured or adapted for use with traditional tattoo tubes and needles, or for use with tube and needle cartridges having an internal membrane or bias element acting on the needle.

### RELATED APPLICATIONS AND CLAIM OF PRIORITY

This patent document claims priority under 35 U.S.C. §119(a) to Canadian patent application number 2915552, filed Dec. 18, 2015.

### FIELD OF THE INVENTION

The invention relates to tattoo machines and specifically, rotary tattoo machines.

### BACKGROUND

Most tattoos are applied by a tattoo artist using a tattoo machine to drive a needle assembly which pierces the skin and delivers ink. The up and down or reciprocating movement of the needle assembly may be achieved using a hand-held coil or rotary tattoo machine. Both types of machines are powered by an external power supply and typically operated by the artist through the use of a foot switch. The needle assembly typically includes multiple needles and the shape, size, and arrangement of needles are selected depending on the effect or design desired for the tattoo. For a rotary tattoo machine, the needle configuration, as well as the configuration of the rotary tattoo machine's motor, stroke wheel, and slide, all may be varied either within a single machine assembly or system, or between different machines. These components are varied to change the give or feel of the machine for different artists and to use the machine for different lining, coloring, or shading steps in the tattooing process. The rotary tattoo machine typically includes a return spring which functions to help return the slide of the machine, and the attached needle, to its resting or up position while the machine is in use, and particularly after use is stopped. Where the needle assembly includes a spring or resilient membrane to return the needle to an up or resting position, the rotary machine may also be configured with the return spring removed.

### SUMMARY

According to an embodiment of the present disclosure there is provided a rotary tattoo machine including a slide member configured to engage a tattoo needle assembly; a body, the body defining a first cavity for slideably engaging the slide member, and the lower portion of the body defining a channel from an exterior surface of the lower portion of the body to the first cavity, the channel being configured to selectively receive a return spring; and a motor coupled to the slide member, the motor causing the slide member to move vertically in the first cavity. In some embodiments the channel extends vertically from a bottom exterior surface of the lower portion of the body to the first cavity. The tattoo machine may include a cap to cover a first end of the channel at the bottom surface of the body. The cap may be a cover, a stopper, a plug or a retaining means, such as set screw or shoulder bolt, which may be used to hold the return spring within the channel. The tattoo machine may include the return spring which is received within the channel to bias the slide to a return position. The return spring may be removed through the first end of the channel and be replaced with a spring with a degree of bias that the artist prefers (which may be softer or stiffer), all while the motor remains coupled to the slide member. Alternatively, for use of the machine with a tube and needle assembly or cartridge having an integrated biasing means to move a needle of the needle assembly to a return position within the tip or cartridge, the return spring may be removed entirely through the first end of the channel while the motor remains coupled to the slide member.

In some embodiments, there is provided a system including the tattoo machine described above, the return spring, a retaining means, a needle assembly and a tube having a stem, a grip and a tip. The system may include a set of return springs, each return spring in the set having a different compression rating. In some embodiments, there is provided a system including the tattoo machine described above, and a stem, a grip, a needle drive bar and a needle cartridge which has a tip and one or more tattoo needles. The cartridge may include a bias means to move a needle of the needle assembly to a return position within the cartridge. Either system may be provided with a set of return springs having a range of compression ratings, or other characteristics, in order to allow a tattoo artist to control and configure the feel and performance of the system.

### BRIEF DESCRIPTION OF THE DRAWINGS

The following detailed description when read in connection with the accompanying drawings describes embodiments of the invention. The various features of the drawings are not necessarily to scale.

FIG. 1 is a side view of a rotary tattoo system as known in the prior art;

FIG. 2A is an exploded, perspective view of a second tattoo machine as known in the prior art and FIG. 2B is an exploded, side, cross-section view of the tattoo machine of FIG. 2A;

FIG. 3 is a top, perspective view of a tattoo machine according to one embodiment of the present disclosure;

FIG. 4 is a bottom, perspective view of a tattoo machine according to one embodiment of the present disclosure;

FIG. 5A is a front, exploded view of a tattoo machine according to one embodiment of the present disclosure

FIG. 5B is a side, cross-section, exploded view of a tattoo machine, along line A-A of FIG. 5A, according to one embodiment of the present disclosure;

FIG. 5C is a side, cross-section, view of a tattoo machine according to one embodiment of the present disclosure;

FIG. 6 is a front view of a tattoo machine body according to another embodiment of the present disclosure; and

FIG. 7A is a front view of a tattoo machine slide according to another embodiment of the present disclosure, FIGS. 7B and 7C are rear and front perspective views of the slide of FIG. 7A.

While the invention will be described in conjunction with the illustrated embodiments, it will be understood that it is not intended to limit the invention to such embodiments. On the contrary, it is intended to cover all alternatives, modifications and equivalents as may be included within the scope of the invention as defined by this specification as a whole, including the appended claims.

### DETAILED DESCRIPTION

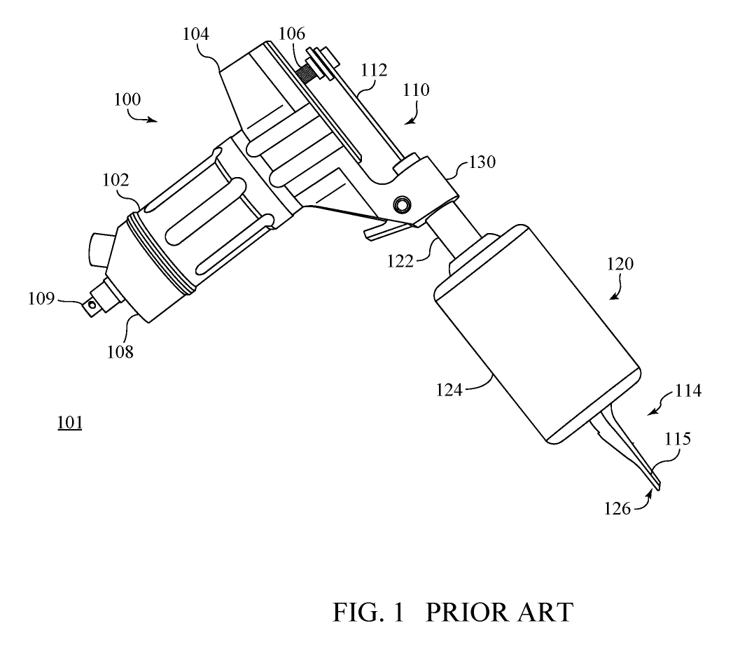

FIG. 1 is a side view of a rotary tattoo machine 100 and tattoo system 101 as known in the prior art. The machine 100 includes a motor 102, a body 104 and a slide 106. The slide 106 is a member which slidably engages the body 104. The slide 106 is configured to receive and retain a tattoo needle assembly 110 or a drive bar for a needle cartridge system as described below. The motor 102 is coupled to the slide 106 through the body 104 of the machine 100, such as with a bayonet type of mount between the motor 102 and the body 104. A first end of the motor 102 may be attachable to the body 104 and the second or opposite end may be covered by a rear cap 108. The rear cap 108 also may include a connection 109 for the power supply (not shown).

The needle assembly 110 includes an upper portion 112 which is configured to engage the slide 106 of the tattoo machine. The lower portion 114 of the needle assembly 110 includes one or more tattoo needles 115. The tattoo needle(s) 115 may be one of hundreds of configurations with different numbers, diameters, shapes, tapers, and groupings of needles. The configuration is selected by the artist depending on the type of tattoo technique being used, which may be drawing a thick or thin line, applying color to an area, or shading an area; and the complexity of the artwork, along with the tattoo artist's personal preferences. Individual artists usually have their own preferences for needle configurations. With some tattoo systems 101, the needle assembly 110 extends through a tattoo tube 120 which is mounted to the tattoo machine 100. The tattoo tube 120 is held by the artist during use. The tattoo tube 120 may consist of a stem 122, a grip 124 and a tip 126. The tube 120 is mounted to the tattoo machine 100 typically by clamping the stem 122 of the tube 120 within an adjustable clamp or vice 130 of the tattoo machine 100. During use, the needle assembly 110 is allowed to move up and down within the tube 120 as the slide 106 is driven by the motor 102. The tattoo machine 100, needle assembly 110, and tube 120 must be precisely configured and assembled, however, to ensure free and consistent movement of the tattoo needle 115 and to allow the tattoo artist to precisely deliver ink to the desired portion of the tattoo design and to the appropriate depth of skin. For health and safety purposes, some or all of the needle assembly 110 and tube 120 must be sterilized or replaced between uses of the system 101 with different tattoo recipients.

The tube 120 also may have various configurations depending on whether the tube 120 is disposable, reusable, or partially re-useable. A disposable tube 120 is intended for one-time use. The tube 120 is typically made of plastic and may consist of a one-piece assembly of the stem 122, grip 124 and tip 126. For a reusable or partially re-useable tube 120, one or more of the components of the tube 120 are typical made of metal and may be separated for sterilization. The configuration of the tube 120, and in particular the grip 124 and tip 126, also may be changed to accommodate different configurations of tattoo needles 115.

In some systems 101, as described above, the needle assembly 110 and the tube 120 are separate components and the needle assembly 110 is a one-piece assembly which moves freely within the tube 120. In other systems (not shown), the needle assembly is comprised of two pieces, an upper portion or drive bar which engages the slide 106 and a lower portion which includes the one or more tattoo needles. The driver bar of the needle assembly is configured to engage the separate lower portion in various ways, such as with a mechanical or magnetic engagement, to ensure that the slide 106 and drive bar control the movement of the lower portion and the one or more tattoo needles 115. With a two-piece type of needle assembly, the lower portion may be combined with one or more components of the traditional tube 120. For example, the lower portion of the needle assembly may be combined with a tip. The combined needle and tip may be referred to as a needle cartridge. The cartridge may be attached to a second assembly consisting of a tube grip and stem, with the stem being held within the vice 130 of the tattoo machine as described above. The needle cartridge may be mounted to or mechanically or frictionally held within the grip. The driver bar of the needle assembly is attached to the slide 106 and extends through the grip and stem to engage the needle cartridge. With a needle cartridge, the one or more tattoo needles and tip typically may be removed from the grip and stem and replaced with a different configuration of the one or more tattoo needles without removing the upper portion of the needle assembly from the slide 106. The cartridge tip may include, for example, a resilient element such as a membrane, elastic band, or spring member to engage the one or more needles and assist with returning the needles to an up or resting position. In this up or resting position, the one or more needles are drawn into the tip 126.

The two-piece type of needle assembly with separate upper and lower portions may enable the tip and needle to be removed from the grip and stem assembly and replaced more easily with a different needle configuration in order to accommodate the different tattoo techniques and factors previously mentioned. In some embodiments, such as where the tattoo machine is configured for the application of permanent make-up to the skin, the cartridge may include an ink reservoir or ink delivery system to avoid having to dip the needle and into a pot or cap of ink during use.

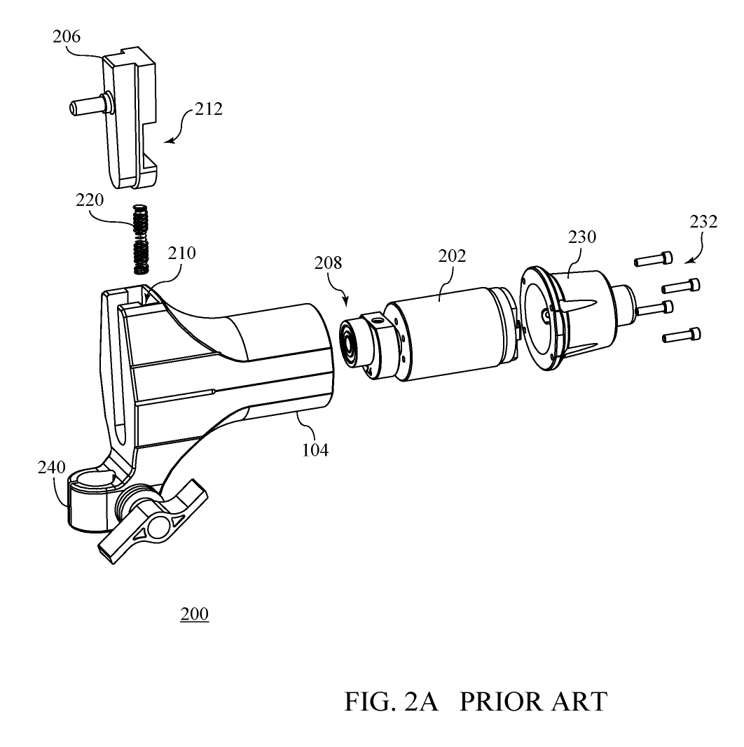

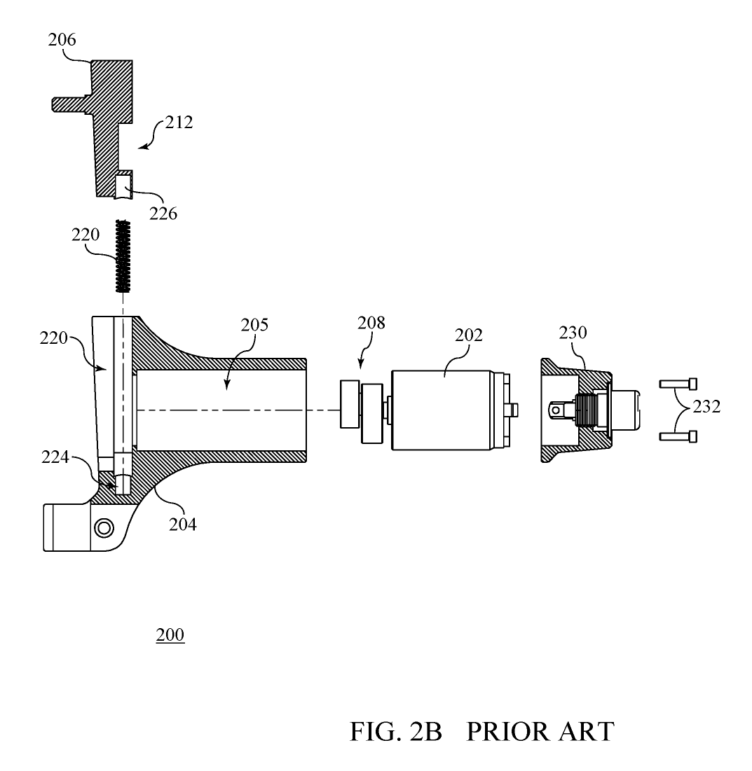

FIGS. 2A and 2B show an exploded view and an exploded cross-section view of a second rotary tattoo machine 200 as known in the prior art. The machine 200 is similar to the machine 100 of FIG. 1 and includes a motor 202, a body 204, and a slide 206. In the machine 200, the body 204 defines a cavity 205 for receiving the motor 202.

In both machines 100, 200, the motor 102, 202 is coupled to the slide 106, 206 through a mechanical linkage. As illustrated in FIGS. 2A and 2B, the mechanical linkage may be a cam assembly 208. The cam assembly 208, which also may be referred to as a stroke wheel, operates to translate the rotary movement of the motor 202 to the reciprocating motion of the slide 206 and the attached needle assembly 110 (not shown in FIGS. 2A and 2B). Different configurations of the cam assembly 208 can be used to control the stroke length of the system. It can be seen from FIGS. 2A and 2B that the slide 206 is received within a cavity 210 defined in a front portion of the tattoo machine body 204. An opening or slot 212 defined at the back of the slide 206 receives a portion of the cam assembly 208 which extends into the cavity 210. The slide 206 is thus controlled by the cam assembly 208 and permitted to move vertically within the cavity 210. Portions of the slide 206 are often lubricated to facilitate this movement.

The tattoo machine 200 includes a return spring 220 which is mounted vertically within the cavity 210 and is retained within the cavity 210 between the body 204 and the slide 206 when the machine 200 is assembled. Although not visible in FIG. 1, the tattoo machine 100 includes a similar return spring retained within a cavity between the body 104 and slide 106 when the machine 100 is assembled. The return spring 220 may be compressed or preloaded in the retained position. Specifically, the return spring 220 is retained within a bottom recess 224 of the cavity 210 and within a cavity 226 defined in a bottom portion of the slide 206 which aligns with the cavity 210 and recess 224. When the machine 200 is assembled, the return spring 220 is retained and may be compressed or preloaded within the cavities 210, 226, and recess 224. The return spring 220 acts against the downward movement of the slide 206 and needle assembly 110 to soften the movement of the tattoo needle 115 and reduce or eliminate vibrations of the machine 200. The return spring 220 also acts to force the slide 206 back to its return position at the top of the cavity 210 during use, and particularly after the motor 202 has been stopped. The return spring 220 also helps to keep the slide 206 in mechanical contact with the cam assembly 208. Both of these actions affect the feel of the machine to the artist. In some systems, the return spring 220 helps to ensure that the tattoo needle 115 is retracted within the tube 120 when the system 101 is not in use.

To assemble the rotary tattoo machine 100 or the machine 200, the return spring 220 may be first inserted in the cavity 226 defined in the bottom portion of the slide 206. The slide 206 and return spring 220 are then lowered and inserted into the cavity 210 in the machine body 204, while aligning the return spring 220 with the bottom recess 224. The motor 202 and cam assembly 208 are then inserted into the body 204 and aligned with the slide 206 and the slot 212. The motor 202 and cam assembly 208 are secured to the body 204, such as by set screws (not shown). The stem portion of the tube (not shown) is mounted and held within an adjustable clamp or vice 240 as described above.

It will be apparent from FIGS. 2A and 2B that in order to remove or replace the return spring 220, a rear cap 230 with set screws 232, the motor 202, the cam assembly 208, and the slide 206 must be removed and replaced. The return spring 220 may be removed or changed, for example, to support different configurations of the needle assembly 110 and tube 120, including various needle cartridges. Alternatively, the return spring 220 may remain in place in the machine 200 when the system is configured with a tube 120 or cartridge having a resilient membrane or spring member which engages the lower portion 114 of the needle assembly 110. The feel of the system may be compromised, additional power may be required, and the machine 200 may suffer additional wear and tear, however, due to having to overcome the resistance of the resilient membrane or spring member during use in addition to the resistance of the return spring 220.

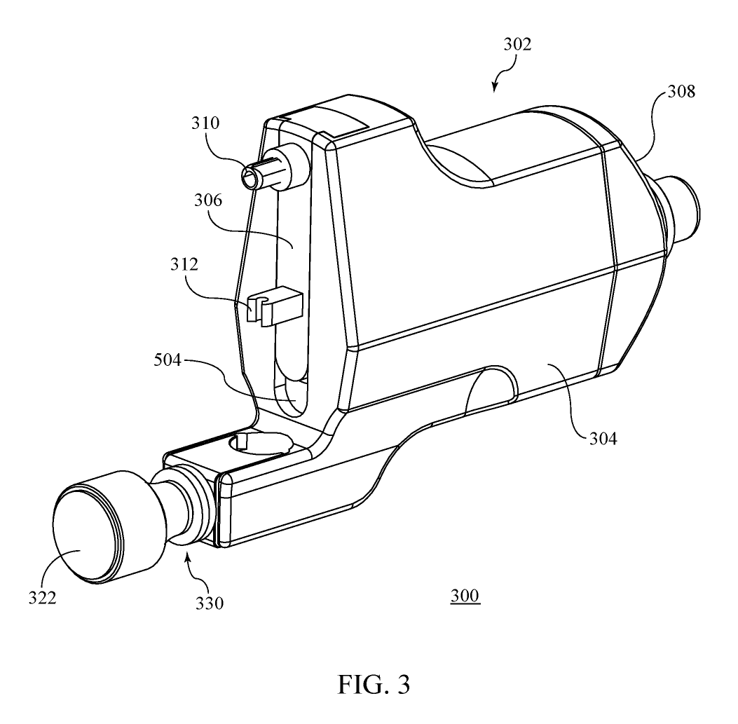

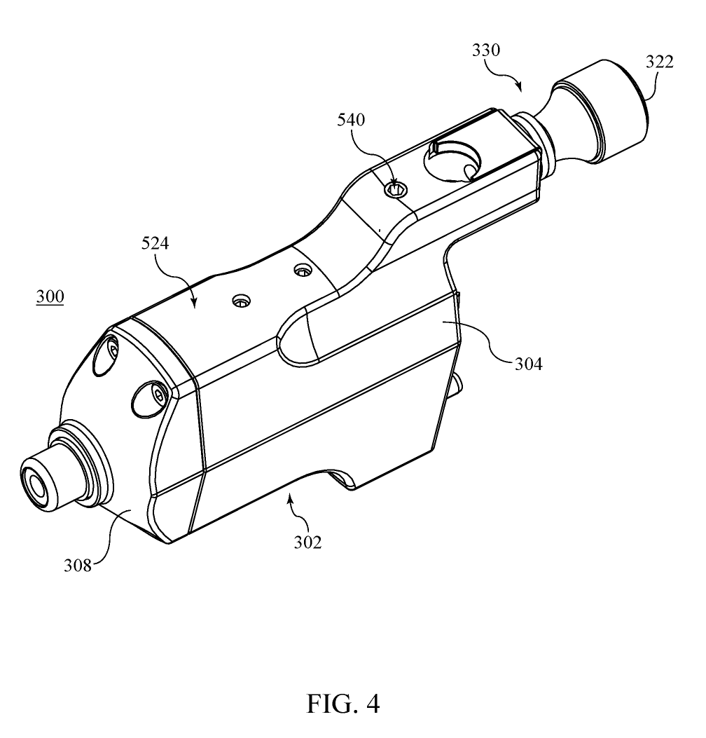

FIGS. 3 and 4 show top and bottom perspective views of a tattoo machine 300 according to an embodiment of the present disclosure. The machine 300 includes a motor 302, a body 304 and a slide 306. The motor 302 is coupled to the slide 306 through the body 304. The motor 302 may be secured by a rear cap 308 and/or a set screw or screws or other clamping mechanisms. The power connection for the machine 300 may be made through the rear cap 308. The slide 306 is a mechanical member which slidably engages the body 304. The slide 306 is configured to receive and retain a tattoo needle assembly 110 (not shown) and to slide vertically with respect to the body 304. The slide 306 may include a pin 310 for receiving and retaining the needle assembly 110. The slide 306 also may include a guide member 312. The guide member 312 extends outward from the slide 306 and is adapted to engage and guide the needle assembly 110 to ensure the needle assembly remains straight during use. The tattoo machine 300 includes a vice 320 for engaging and securing the stem 122 of the tattoo tube 120. The vice 320 may consist of a clamp or other configuration to engage and hold the stem 122 of the tattoo tube 120. The vice 320 may be adjusted such as by the use of a thumbscrew 322 to secure and release the tube 120. The tattoo machine 300 is configured to accommodate the needle assembly 110 and tube 120, which, as described above may include a number of different components and arrangements. The needle assembly 110 may include a one piece arrangement or separate upper and lower portions. In the latter case, the lower portion of the needle assembly may be provided in a needle cartridge as described above, with or without an ink reservoir and delivery system. The cartridge may include a biasing means, such as a resilient membrane or spring member which engages the lower portion of the needle assembly 110 and forces the one or more tattoo needles to return to an up or resting position.

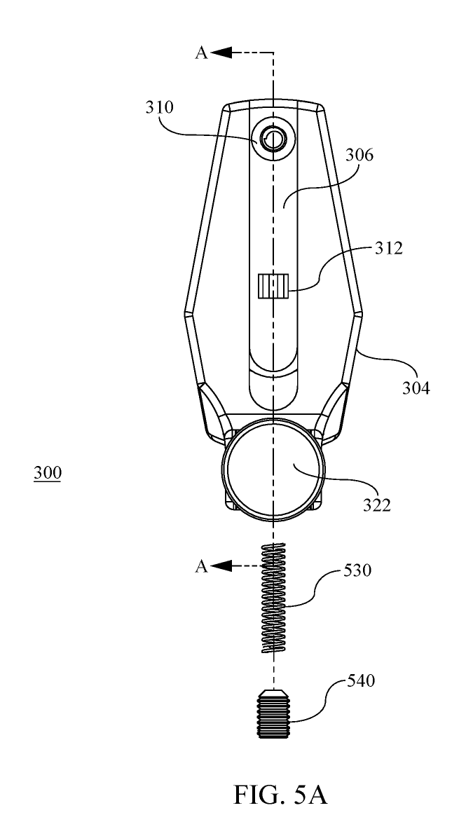

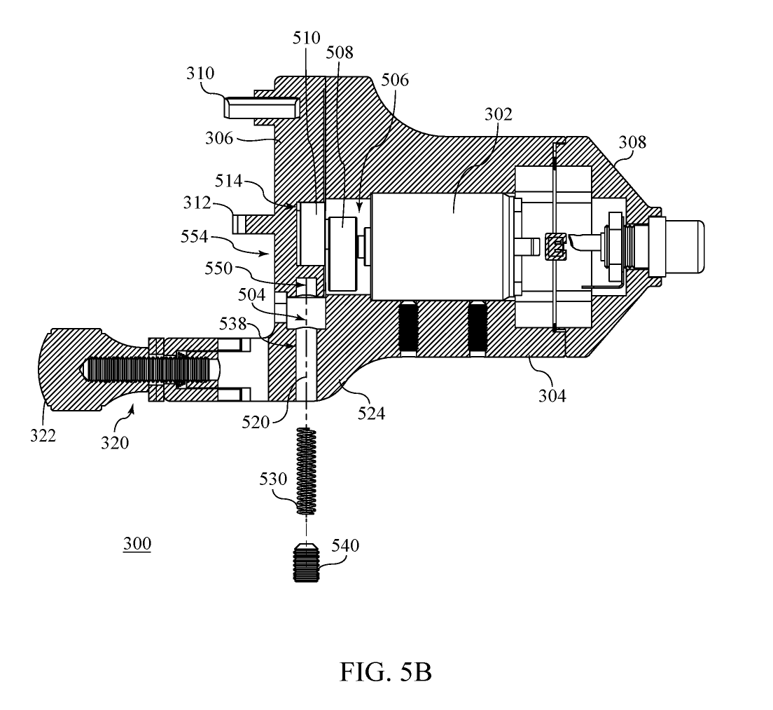

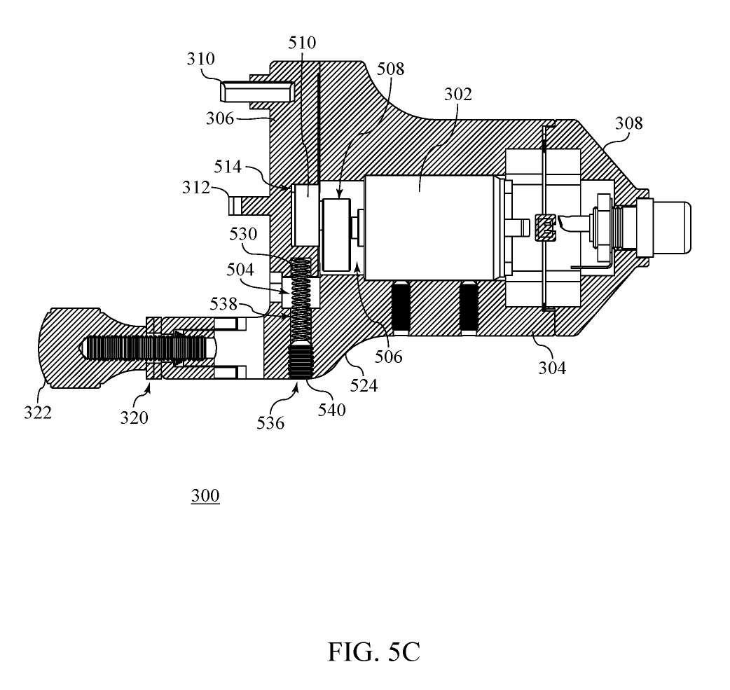

As illustrated in FIGS. 5A to 5C, in one embodiment, the body 304 of the tattoo machine 300 defines a first cavity 504 which is configured to receive and engage the slide 306. The first cavity 504 may be in communication with a second cavity 506 which receives the motor 302. In other embodiments, not shown, the motor 302 is not housed within the body 304 but may be coupled to the body 304 with a bayonet type of mount. The first cavity 504 is in communication with the second cavity 506 through the attachment of the motor 302 to the body 304.

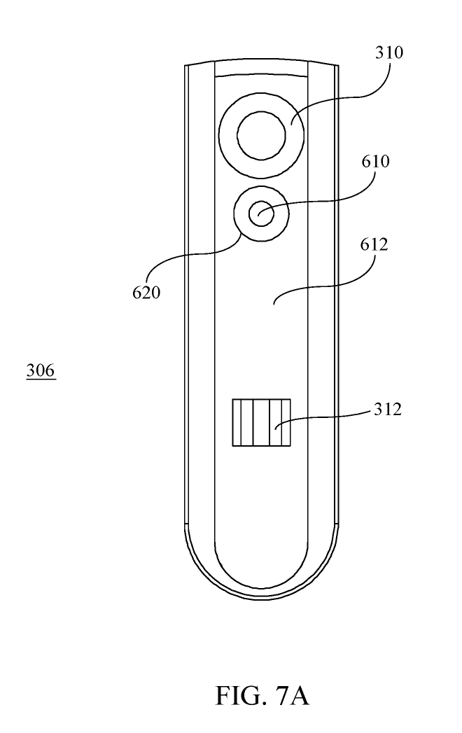

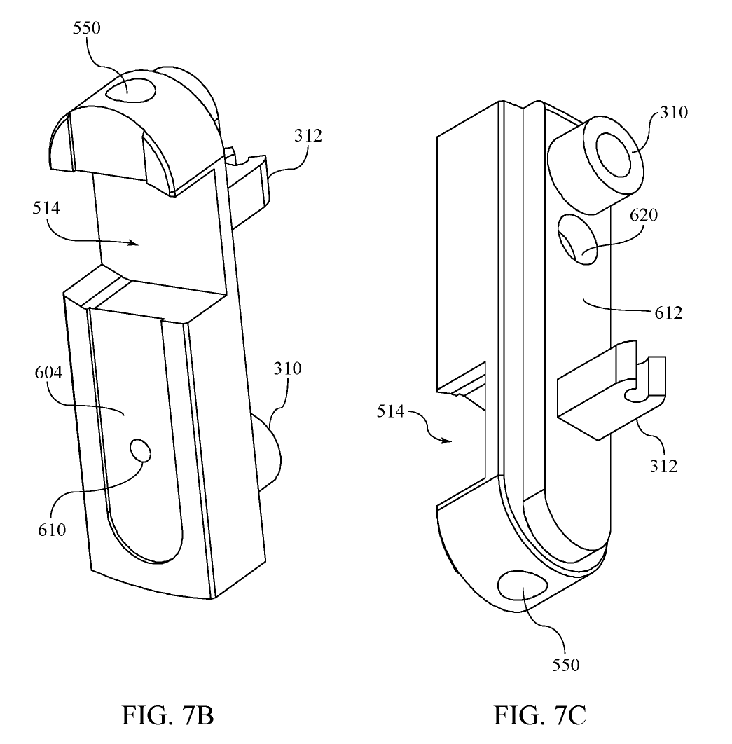

The motor 302 is typically coupled to the slide 306 through a mechanical linkage such as a cam assembly 508. The cam assembly 508 operates to translate the rotary movement of the motor 302 to the reciprocating motion of the slide 306 and the attached needle assembly 110. The cam assembly 508 includes a cam bearing 510 which extends into the first cavity 504 to engage a slot 514 defined in the slide 306. The slot 514 can be seen more clearly in the embodiments show in FIGS. 7-7C, as described below. In other embodiments (not shown), the mechanical linkage may consist of a linkage arm coupled to the slide 106 or an oval shaped cam assembly.

The body 304 defines a channel 520 which extends from an exterior surface in the lower portion of the body to the first cavity 504. In one embodiment, the channel 520 extends from a bottom exterior surface 524 of the body 304 through to the first cavity 504. The channel 520 may be defined substantially vertically within the body 304 in order to align with the slide 306 when the slide 306 is assembled within the cavity 504. In some embodiments, where the slide 306 and first cavity 504 are angled slightly within the tattoo machine 300, at least an upper portion of the channel 520 may be angled to align with the slide 306. In other embodiments (not shown), a portion of the channel 520 may be curved, angled, or multi-angular and may extend from a side exterior surface or an angled bottom exterior surface of the lower portion of the body 304 to the first cavity 504. It will be appreciated that the channel 520, and access to the channel 520, may be provided in a number of ways through the lower portion of the body 304.

The channel 520 is configured to selectively receive a return spring 530. The return spring 530 may be inserted or removed from the machine 300 through a first end 536 of the channel 520 at the bottom exterior surface 524 of the body 304. In some embodiments, when the machine 300 has been disassembled, the return spring 530 may be inserted into the body 304 through a second end 538 of the channel 520 adjacent the first cavity 504. It will be appreciated, however, that with the configuration of the machine 300 and channel 520, the return spring 530 may be preferably inserted or removed from the machine 300 through the first end 536 of the channel 520 without disassembling the machine 300 and without decoupling the motor 302 from the slide 306. The return spring 530 thus may be selectively removed or provided within the channel 520 while the slide 306, motor 302 and body 304 components remain assembled. The return spring 530 also may be selectively removed or provided within the channel 520 while the needle assembly and tube (not show) remain mounted to the tattoo machine 300.

When the machine 300 is used with a needle assembly and tube or cartridge having a biasing means to force the one or more tattoo needles to an up or return position, the return spring 530 may be removed. In other embodiments, for any type of tube and needle assembly or cartridge, the return spring 530 may be removed and substituted with a different return spring 530. Thus, different return springs 530 may be selectively placed within the channel 520 to alter the performance, use and feel of the tattoo machine 300. In some embodiments, the use of a selected return spring 530 may reduce the power required from the motor 302 for a particular step. The machine 300 essentially works less to produce a particular action which reduces the wear and tear for the machine 300.

The resistance of the return spring 530 thus can be easily removed if it is redundant in light of the bias provided in a needle cartridge. The resistance of the return spring 530 also can be easily changed to complement any bias provided in the needle cartridge. The return spring 530 may be selected depending on a number of factors including, but not limited to, the artist's preference or feel and tolerance for noise and vibrations; the degree of return bias (if any) in the needle cartridge or needle assembly itself; the power provided by the motor 302; the configuration and size of the slide 306 and cam assembly 508; and the type of tattoo technique and/or design. The selection of the return spring 530 also may depend on the fit and tolerances between the cam bearing 510 and the slot 514 in the slide 306.

As one example, a lining tattoo action is typically done at higher speeds and voltages. With machines known in the prior art, this may result in vibration and a harsh feel or sense of needle hit. The return spring provided in these machines typically is stiff enough to accommodate this range of operations and higher speeds and voltages even though performance, power consumption, and the feel of the machine may be comprised for other actions. An artist may choose to operate the machine at a lower speed where a harsh feel does not pose a problem, but power consumption is likely higher as a result, and the machine may feel sluggish.

With the features disclosed herein, an artist may easily configure the machine 300 with a different return spring 530 and operate the machine 300 at a lower power setting. For example, while a particular return spring 530 may be sufficient for a motor 302 operating at 100 rpm (revolution per minute), a speed commonly used for a coloring tattoo action, a softer return spring 530 with less resistance may be used in order to operate the machine 300 at the same rpm but with less current. At higher rpms, such as those used for the actions to draw thin lines for a tattoo, a stiffer return spring 530 with greater resistance may be chosen in order to have the machine 300 run more efficiently. By providing access to the channel 520 through the lower portion of the tattoo machine body 304, the return spring 530 may be quickly and easily changed without disassembling the machine 300. Rather than providing a return spring 530 which is sufficient for a majority of tattooing actions and machine and needle configurations, the machine 300 may be operated with more efficient power consumption by using a return spring 530 which matches, among other factors, the rpms used for the tattooing action.

When the return spring 530 is removed from the channel 520, a cap 540 may be used to cover or close the first end 536 of the channel 520 at the bottom surface 524 of the body 304. Although the channel 520 does not include the return spring 530 in this configuration, the cap 530 may be provided to prevent the channel 520 from being contaminated during the tattooing process. The cap 530 may be a cover, a stopper, a plug, a set screw, a shoulder bolt or any other means to close or seal the first end 536 of the channel.

In other embodiments, as described above, the return spring 530 may be included or configured as part of the machine 300. In this case, the cap 530 may be a retaining means to hold the return spring 530 within the channel 520. Specifically, the retaining means may be configured to hold or bias the return spring 530 against the slide 306. In some embodiments, at least a portion of the interior surface of the channel 520 may be threaded and configured to receive a retaining means such as a set screw or shoulder bolt. In some embodiments, the retaining means provides a cover for the first end 536 of the channel 520 as well as providing a lock to ensure the retaining means and return spring 530 remain in place despite the movement of the slide 306 and vibrations of the machine 300 during use. In some embodiments, the retaining means may be dimensioned or configured to provide a specific vertical placement and preload of the return spring 530.

In some embodiments, the slide 306 defines a recess 550 in a bottom or lower portion 554 of the slide 306. The recess 550 is configured to receive a top or upper portion of the return spring 530. The recess 550 is aligned with at least an upper portion of the channel 520.

The channel 520, return spring 530 and recess 550 may be circular or cylindrical in shape although other shapes and configurations may be used. Similarly, the cap 530 or retaining means may be circular or cylindrical in shape as shown in FIGS. 5A-5C, but other shapes and configurations may be used.

In one embodiment, the return spring 530 is a coiled spring element configured to fit within the channel 520. In some embodiments, the machine 300 may be provided as part of a system including a set of return springs 530. Each spring in the set may be configured with a different compression rating to allow the tattoo artist to select a return spring 530 and alter the tension in the machine 300 and tattoo machine system. In one embodiment, the return springs 530 in the set may have a compression rating varying between 0.8 to 2.8 lbs/inch. The different return springs 530 may be color coded for easier identification. In some embodiments, the return spring 530 may have a variable pitch, also known as a variable rate spring, which provides a different compression rating at different deflections of the spring. This type of return spring may allow the tattoo artist to adjust the spring to be stiffer or softer by changing its preload length (i.e. its deflection) in the channel 520. Alternatively, or in addition, a set of retaining means may be provided with each retaining means in the set being dimensioned to change the position and preload of the return spring 530. Thus, depending on the desired configuration of the slide 306, cam assembly 508, and motor 302, for a particular tattoo design or artist, and a particular lining, coloring or shading step, a different return spring 530 and different retaining means may be selected and installed to change the feel of the system with the tattoo machine 300. As noted above, the return spring 530 may be replaced by removing the cap 540 or retaining means and return spring 530, but without disassembling the motor 302, body 304 and slide 306.

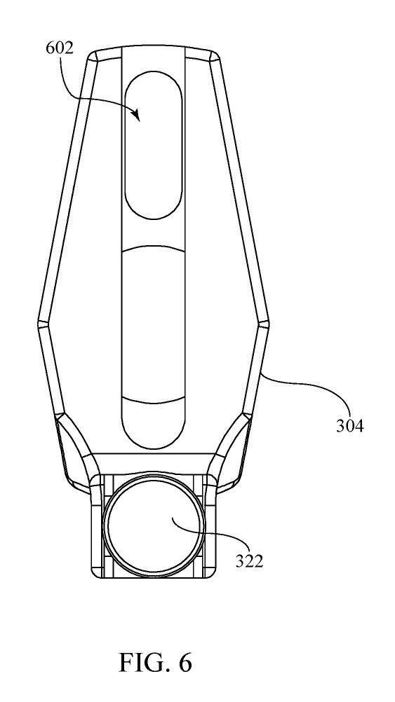

In further embodiments, as illustrated in FIGS. 6, 7A, 7B, and 7C, the slide 306, the body 304, or both the slide 306 and body 304 may be configured to provide access points for lubrication of the slide 306 without disassembling the tattoo machine 300. FIG. 6 illustrates a front view of the body 304 of the tattoo machine 300 with the slide 306 removed. The body 304 further defines a small recess 602 in a front exterior surface of body 304 within the first cavity 504. The recess 602 may be oval, rectangular, or circular in shape. The recess 602, in some embodiments, is located about 2 mm below the top of the body 304 and is about 14 mm long and 5 mm wide. In some embodiments, the recess 602 is about 0.5 mm deep providing a shallow pool or cavity for holding lubricant for the slide 306. The recess 602 is provided in the upper portion of the first cavity 504 to prevent lubricant from seeping towards the motor 302 or into the second cavity 506 containing the motor 302. When the slide 306 is assembled with the body 304, the recess 602 overlaps at least partially with a rear surface 604 of the slide 306 above the slot 514. In some embodiments, a portion of the rear surface 604 also may be recessed slightly, as illustrated in FIG. 7B, to retain lubricant for the slide. It will be appreciated that when the slide 306 is in a down position, i.e. at the bottom of the stroke of the tattoo needle, at least a portion of the recess 602 in the body 304 is visible and accessible at the front of the tattoo machine 300 for adding lubricant for the slide 306.

In some embodiments, the slide 306 defines a lubricant channel 610 which extends from a front surface 612 of the slide 306 to the rear surface 604. The lubricant channel 610 is provided to receive and deliver lubricant to the rear surface 604 of the slide 306 and to one or more surfaces of the first cavity 504 of the body 304. The lubricant channel 610 may be circular and may range in width from 0.76 to 1.7 mm. The lubricant channel 610 may be dimensioned to accommodate different types of lubricant for the slide 306, including grease or oil. The lubricant channel 610 may be located below the pin 310 and may extend perpendicularly through the slide 306. In some embodiments, the location and angle of the lubricant channel 610 may be varied in order to provide an access point at the front of the slide 306 which allows access to the recess 602 in the body 304. In some embodiments, the lubricant channel 610 may have a stepped configuration with a larger channel 620 adjacent the front surface 612 of the slide 306. The larger channel 620 improves access to the lubricant channel 610 for injecting the lubricant. In other embodiments (not shown), the lubricant channel 610 may extend to openings on one or both sides of the slide 306 instead of, or in addition to, extending to the rear surface 604 of the slide 306.

Although lubricant may be added when the slide 306 is changed, such as to configure the machine 300 with a different slide 306, the recess 602 and lubricant channel 610 allow lubricant to be added without removing the slide 306 from the machine 300.

Although the present disclosure describes methods and processes with steps in a certain order, one or more steps of the methods and processes may be omitted or altered as appropriate. One or more steps may take place in an order other than that in which they are described, as appropriate.

While the invention has been described in conjunction with illustrated embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention. In particular, features from one or more of the above-described embodiments may be selected to create alternate embodiments comprised of a subcombination of features which may not be explicitly described above. In addition, features from one or more of the above-described embodiments may be selected and combined to create alternate embodiments comprised of a combination of features which may not be explicitly described above. Features suitable for such combinations and subcombinations would be readily apparent to persons skilled in the art upon review of the present application as a whole. Any dimensions provided in the drawings are provided for illustrative purposes only and are not intended to be limiting on the scope of the invention. The subject matter described herein and in the recited claims intends to cover and embrace all suitable changes in technology.

1. A rotary tattoo machine comprising:
a slide member configured to engage a tattoo needle assembly;
a body, the body defining a first cavity for slideably engaging the slide member, and the lower portion of the body defining a channel from an exterior surface of the lower portion of the body to the first cavity, the channel being configured to selectively receive a return spring; and
a motor coupled to the slide member, the motor causing the slide member to move vertically in the first cavity.

2. The rotary tattoo machine of claim 1 wherein the channel extends vertically from a bottom exterior surface of the lower portion of the body to the first cavity.

3. The rotary tattoo machine of claim 1 further comprising a cap to cover a first end of the channel at the bottom surface of the body.

4. The rotary tattoo machine of claim 3 wherein the machine includes the return spring received within the channel to engage and bias the slide member to a return position and wherein the cap comprises a retaining means to hold the return spring within the channel.

5. The rotary tattoo machine of claim 4 wherein the retaining means holds the return spring against the slide member.

6. The rotary tattoo machine of claim 5 wherein the slide member defines a recess in a bottom portion of the slide member, the recess being aligned with the channel to receive a top portion of the return spring.

7. The rotary tattoo machine of claim 4 wherein the return spring is removable from the channel through the bottom surface of the body while the motor remains coupled to the slide member.

8. The rotary tattoo machine of claim 4 wherein the return spring comprises a variable pitch spring.

9. The rotary tattoo machine of claim 4 wherein the channel is internally threaded and wherein the retaining means comprises a set screw or a shoulder bolt.

10. The rotary tattoo machine of claim 4 wherein each of the channel and return spring has a cylindrical shape.

11. The rotary tattoo machine of claim 2 wherein the cap comprises a cover, a stopper, a plug, a set screw, or a shoulder bolt to close the first end of the channel.

12. The rotary tattoo machine of claim 1 wherein the slide member further defines a channel from a front surface of the slide member to a rear surface of the slide member, the channel being configured to receive and deliver lubricant for the slide member and the first cavity of the body.

13. The rotary tattoo machine of claim 1 wherein the body further defines a recess in a front exterior surface of the first cavity, the recess being configured to receive lubricant for the slide member and wherein at least a portion of the recess is accessible when the slide member is in a down position.

14. A system comprising the rotary tattoo machine of claim 4, the needle assembly, and a tattoo tube comprising a stem, a grip and a tip, the tattoo tube being mounted to the tattoo machine and configured to receive the needle assembly.

15. The system of claim 14 further comprising a set of return springs, each return spring in the set having a different compression rating.

16. A system comprising the rotary tattoo machine of claim 1, a stem, a grip, a needle drive bar and a needle cartridge comprising a tip and one or more tattoo needles, the cartridge having a biasing means to move the one or more needles to a return position within the cartridge.

17. The system of claim 16 further comprising a cap to cover a first end of the channel at the bottom surface of the body.

18. The system of claim 17 wherein the cap comprises a cover, a stopper, a plug, a set screw, or a bolt screw to close the first end of the channel.

19. The system of claim 16 wherein the rotary tattoo machine includes the return spring received within the channel to engage and bias the slide member to a return position, wherein the cap comprises a retaining means to hold the return spring within the channel, and wherein the system further comprises a set of return springs, each return spring in the set having a different compression rating.
